(19) United States
(12) Patent Application Publication
(10) Pub. No.: US 2008/0108709 A1
(43) Pub. Date: May 8, 2008

(54) COLD-PREPARABLE, LOW-VISCOSITY AND PROLONGED COSMETIC EMULSIONS WITH COEMULSIFIERS CONTAINING CATIONIC GROUPS

(75) Inventors: Jurgen Meyer, Munster (DE); Burghard Gruning, Essen (DE); Peter Hameyer, Essen (DE); Holger Leidreiter, Hattingen (DE); Hans Henning Wenk, Essen (DE)

Correspondence Address: SCULLY SCOTT MURPHY & PRESSER, PC, 400 GARDEN CITY PLAZA, SUITE 300, GARDEN CITY, NY, 11530, US

(73) Assignee: GOLDSCHMIDT GMBH, Essen (DE)

(21) Appl. No.: 11/869,456

(22) Filed: Oct. 9, 2007

(30) Foreign Application Priority Data
Oct. 6, 2006 (DE) 10 2006 047 247.0

(51) Int. Cl. A61K 47/00; A61K 47/26; A61K 47/12; A61K 47/18; A61K 47/14

(52) U.S. Cl. 514/777; 514/772; 514/784; 514/788; 514/785

(57) ABSTRACT

PEG-free, cold-preparable, prolonged-stability, low-viscosity, fine oil-in-water emulsions, their preparation from preferably clear oil phases or via preferably clear to transparent microemulsion-like concentrates, the corresponding oil phases or microemulsion-like concentrates and the use of the inventive emulsions for producing cosmetic, dermatological, pharmaceutical or industrial formulations, especially for the production of impregnating emulsions for wet wipes or for sprayable care emulsions are provided.

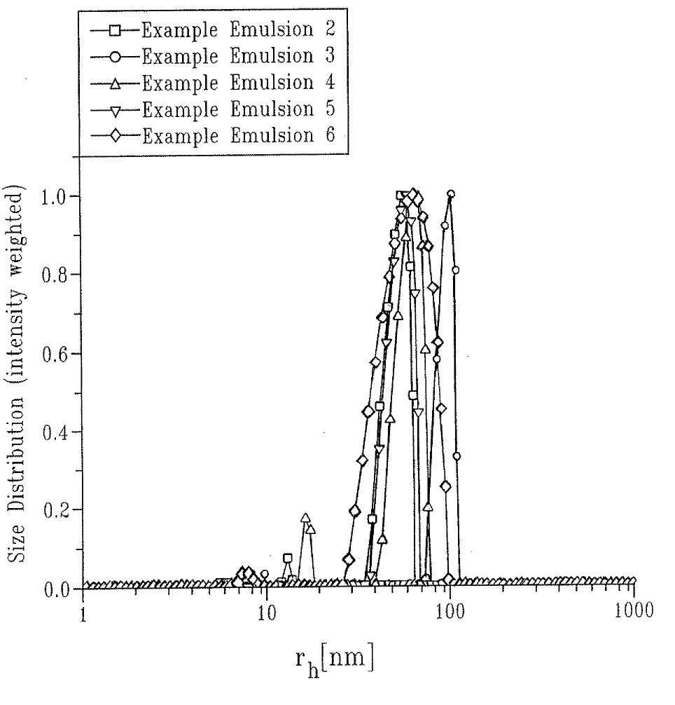

### FIELD OF THE INVENTION

The present invention provides cold-preparable, prolonged-stability, low-viscosity, fine oil-in-water emulsions. The present invention also provides a method for the preparation of such emulsions from preferably clear oil phases or via preferably clear to transparent microemulsion-like concentrates. The present invention also relates to the corresponding oil phases or microemulsion-like concentrates and the use of the inventive emulsions for producing cosmetic, dermatological or pharmaceutical formulations, and for the production of cleaning and care emulsions for the household and industry, especially for the production of impregnating emulsions for wet wipes or for sprayable care emulsions.

### BACKGROUND OF THE INVENTION

Emulsions constitute an important product type in the field of cosmetic, dermatological and/or pharmaceutical formulations. Cosmetic formulations are utilized essentially for skincare. Skincare in the cosmetic sense is primarily the enhancement and/or reestablishment of the natural function of the skin as a barrier against environmental influences (for example, soil, chemicals, microorganisms) and against the loss of endogenous substances (for example, water, natural fats, electrolytes).

A further aim of skincare is to compensate for the loss of fats and water in the skin caused by daily washing and to preserve and restore the softness and smoothness of the skin. This is important when the natural regeneration capacity is insufficient. Moreover, skincare products should provide protection from environmental influences, especially from the sun and the wind, and delay skin ageing.

Pharmaceutical topical compositions generally comprise one or more medicaments in effective concentration. For the sake of simplicity, cosmetic and medical use, and corresponding products, are clearly distinguished by reference to the legal stipulations of the Federal Republic of Germany (for example, Cosmetics Act, Food and Drug laws).

In the last few years, cosmetic wet wipes have gained increasing significance because of their extremely simple and convenient usability. Initially, virtually exclusively wet wipes for cleaning purposes were represented on the cosmetics market, which comprised mainly aqueous, surfactant-containing impregnating solutions. However, in recent times, care products have also been appearing more and more on the market, which are based on impregnating emulsions and thus additionally comprise a care oil component.

Most of these cosmetic wet wipes for bodycare and facecare are impregnated with emulsions which have been prepared by the PIT emulsifying method (such as described, for example, in K. Shinoda, H. Kunieda, Phase properties of emulsions: PIT and HLB, Encycl. of Emulsion Technology, 337-367 (1), 1983 or Th. Förster, F. Schambil, W. von Rybinski, J. Disp. Sci. And Technology, 13(2), 183-93 (1992)).

The PIT method makes use of the fact that, in an oil-in-water (O/W) emulsion which is stabilized by nonionic emulsifiers containing polyethylene glycol (“PEG-containing emulsifiers”), a phase inversion can be induced to give a water-in-oil (W/O) emulsion by increasing the temperature (phase inversion; PIT: phase inversion temperature).

Since the water-oil interface tension is extremely low in this phase inversion region, extremely fine oil-in-water emulsions can thus be obtained after cooling. For this purpose, it is, on one hand, necessary that the individual components of the emulsions are adjusted precisely with respect to one another for each system to be emulsified. This means that emulsifier mixtures and emulsifier concentration have to be “tailored” for different oil phases.

The fine and low-viscosity emulsions thus produced have excellent long-term stability and are thus very suitable as impregnating solutions for wet wipes. Such systems are described, for example, in EP-B-1 268 740 or WO-A-00/04230.

On the other hand, a fundamental requirement in PIT emulsification technology is the necessity for the entire emulsion to be heated to temperatures above the phase inversion temperature and then to be cooled down again.

At the present time, where process operations have to be optimized and energy costs restricted, this means a distinct disadvantage compared to systems which do not have to pass through this heating/cooling curve.

For this reason, fine, prolonged-stability emulsions which can be prepared at room temperature (“cold preparation”) without having to pass through an additional heating/cooling curve would be advantageous.

Another disadvantage in impregnating solutions for wet wipes based on PIT emulsions is that the impregnating solutions are based on the use of PEG-containing emulsifiers. The reason for this is that generally only ethoxylated emulsifiers have as strong a temperature dependence of their hydrophilicity as required for temperature-controlled phase inversion operations.

In view of very natural cosmetic formulations, it is an important aim of cosmetics research to be able to dispense with emulsifiers containing polyethylene glycol (“PEG”). There is therefore an increased search for PEG-free alternative solutions.

It is also known that ethoxylated emulsifiers impart a rather watery skin feel, which can be improved sensorily by the use of, for example, polyglyceryl esters.

For instance, WO-A-02/056841 describes PEG-free impregnating emulsions for cosmetic wet wipes based on polyol poly-12-hydroxystearates and alkyl glycosides. The use of these emulsifier mixtures leads to improved softness of paper products impregnated with them and also leads to improved sensory properties in use of the wet wipes produced with such emulsifier mixtures. In the case of such emulsifier combinations, it is, however, generally difficult to achieve good long-term stability of the impregnating emulsions in combination with sufficient preservation.

Especially in the production of wet wipes, sufficient preservation of the impregnating solutions is absolutely necessary in order to prevent germ growth. The preservation has to be sufficient to protect both the impregnating solutions themselves as well as the impregnated wet wipes in the long term against germ growth.

Preferred preservative mixtures used are typically mixtures of alkylparaben esters and phenoxyethanol, as are commercially available, for instance, under the trade names Buxyl® K 300 (Schüllke & Mayr) or Phenonip® (Clariant).

The described high requirements regarding reliable preservation of impregnating solution and wet wipes make it necessary that relatively large amounts of these alkylparaben ester/phenoxyethanol mixtures generally have to be used in the finished impregnating solutions (0.5 to 1.0% by weight).

It is known that the use of these alkylparaben ester/phenoxyethanol mixtures has an emulsion-stressing influence, since these compounds are very interface-active and compete with emulsifier molecules for a space at the oil-water interface. Because of the interface-active character of these preservative mixtures, the preservative mixtures can also be described as aromatic cosurfactants with preservative properties. In the case of impregnating emulsions for wet wipes, this emulsion-stressing effect is generally enhanced by the required high amounts of these preservatives and the low viscosities of the impregnating solutions.

In summary, it can therefore be stated that it is not possible with the emulsifiers or emulsifier combinations described in the prior art to prepare cold-preparable, sufficiently preserved, low-viscosity, fine and prolonged-stability emulsions, as are typically used for impregnating emulsions or sprayable lotions.

### SUMMARY OF THE INVENTION

The present invention provides a method to prepare low-viscosity, fine and prolonged-stability emulsions, as used typically for impregnating emulsions or sprayable lotions, i.e., which simultaneously
can be prepared at room temperature,
are free of ethoxylated constituents, and
which can readily additionally contain a sufficient amount of preservative compounds.

It has now been found that, surprisingly, low-viscosity, fine and prolonged-stability oil-in-water emulsions, which are outstandingly suitable for use as impregnating emulsions or in sprayable systems, are preparable at room temperature when a suitable combination of emulsifiers based on polyol partial esters and emulsifiers containing catinic groups, cosmetic oils and cosurfactants is used.

The inventive fine oil-in-water emulsions are preferably free of polyethylene glycol-containing substances (“PEG-free”) and comprise an emulsifier mixture consisting of a primary nonionic emulsifier component, preferably polyol partial esters, and a secondary emulsifier component containing cationic groups, and additionally interface-active cosurfactants, and additionally oils, preferably ester and/or ether oils and/or paraffin oils, and optionally further customary assistants and additives.

In addition to their ease of preparation and their extremely fine dispersion, the inventive emulsions are notable in that they are essentially free of ethoxylated ingredients (“PEG-free” emulsion systems). The wet wipes produced with the aid of these impregnating solutions are additionally notable for exceedingly pleasant sensory properties. The emulsifiers containing cationic groups used can both cause a softening effect on the wet wipes and lead to better adhesion of the impregnating emulsion to skin and hair.

The inventive oil-in-water emulsions provide for the first time PEG-free, low-viscosity and fine emulsions which contain cationic groups and are easily preparable at room temperature and can also simultaneously be preserved easily and have prolonged stability by virtue of the use, preferred in accordance with the invention, of preservation-active aromatic cosurfactants, and are thus suitable for use as impregnating emulsions for wet wipes.

The invention therefore provides prolonged-stability, low-viscosity, fine oil-in-water emulsions comprising:
A) an emulsifier mixture consisting of:
   a) at least one nonionic primary emulsifier, and
   b) at least one secondary emulsifier containing cationic groups;
B) one or more cosurfactants;
C) one or more oils; optionally
D) one or more polar solubilizers; and optionally
E) customary assistants and additives,
   with the proviso that the water phase content of the emulsion mixture is ≧70% by weight based on the overall emulsion.

### BRIEF DESCRIPTION OF THE DRAWINGS

The sole FIGURE of the present application is a graph illustrating the intensity-weighted radius distribution of selected emulsions in accordance with the present invention.

### DETAILED DESCRIPTION OF THE INVENTION

As stated above, the present invention provides prolonged-stability, low-viscosity, fine oil-in-water (O/W) emulsions which are PEG-free. The inventive (O/W) emulsion includes:
A) an emulsifier mixture consisting of:
   a) at least one nonionic primary emulsifier, and
   b) at least one secondary emulsifier containing cationic groups;
      one or more cosurfactants; and
      one or more oils, with the proviso that the emulsifier mixture has a water phase content that is ≧70% by weight based on the overall emulsion.

In some embodiments of the present invention, the inventive (O/W) emulsion further includes, as optional components, at least one of the following:

one or more polar solubilizers; and

customary assistants and additives.

Emulsions preferred in accordance with the invention have low viscosity, are finely distributed and have long-term stability.

“Low viscosity” is understood to mean a viscosity which enables spraying of the emulsions with a customary apparatus. In general, these are emulsion viscosities of ≦4000 mPas (Brookfield RVT, spindle 4, 10 rpm (20° C.)), preferably ≦2500 mPas, and more preferably ≦1000 mPas. Higher viscosities are attainable, but not preferred, in accordance with the invention.

“Fine” is understood to mean a mean radius of the emulsion droplets of from ≧20 to ≦500 nm, preferably of from ≧25 to ≦200 nm, and more preferably of from ≧30 to ≦120 nm.

“Prolonged stability” is understood to mean that the inventive emulsions can be stored for three months at room temperature and for 1 month at 40° C. without irreversible creaming or other signs of instability.

The inventive oil-in-water emulsions typically have a water phase content of from ≧70 to ≦99% by weight, and preferably of from ≧80 to ≦97% by weight.

The water phase includes all substances in a formulation which are added to this phase or can be dissolved or dispersed in it owing to their hydrophilic character. Based on the inventive oil-in-water emulsions, water or any constituents such as glycols, polyalkylene glycols, glycerol, polyglycerols, alcohols, water-soluble polymers or active ingredients, in any case, belong to the water phase.

In all embodiments of the invention, the emulsifier mixture (A), the cosurfactants (B) and the oils (C) are used preferably in proportions by weight (based on these three components) of (A) ≧0 to ≦30/(B)≧1 to ≦20/(C) ≧50 to ≦89 and more preferably in proportions by weight of ≧20 to ≦25/≧3 to ≦15/≧60 to ≦77, the emulsifier mixture (A) being composed of from ≧75 to ≦99.9% by weight of nonionic primary emulsifier (a) and from ≧0.1 to ≦25% by weight of secondary emulsifier (b) containing cationic groups.

For the emulsifier mixture (A), preference is given in accordance with the invention to using, for the nonionic primary emulsifiers (a), polyol partial esters selected from at least one of the groups of:

a1) glyceryl and polyglyceryl partial esters, preferably prepared by esterifying aliphatic, linear or branched, optionally unsaturated and/or hydroxy-functionalized carboxylic acids having a chain length of from ≧6 to ≦22 carbon atoms with glycerol, polyglycerols or mixtures of the two,

a2) sorbitan or sorbitol partial esters, preferably prepared by esterifying aliphatic, linear or branched, optionally unsaturated and/or hydroxy-functionalized carboxylic acids having a chain length of from ≧6 to ≦22 carbon atoms with sorbitol,

a3) carbohydrate esters, preferably glycoside or sucrose esters, preferably prepared by esterifying aliphatic, linear or branched, optionally unsaturated and/or hydroxy-functionalized carboxylic acids having a chain length of from ≧6 to ≦22 carbon atoms with mono-, oligo- or polysaccharides, and optionally higher saccharides,

a4) (alkylpoly)glycosides, preferably prepared by reacting aliphatic, linear or branched, optionally unsaturated and/or additionally hydroxy-functionalized alcohols having a chain length of from ≧6 to ≦22 carbon atoms with mono- or polysaccharides.

Mixtures of a1)-a4) are also contemplated within the present invention.

Typically, the nonionic primary emulsifier component (a) is based on polyglyceryl esters, to which sorbitan esters have preferably been added, in an amount of from ≧0 to ≦75% by weight, preferably from ≧0 to ≦50% by weight more preferably from ≧0 to ≦25% by weight, based on the overall primary emulsifier component (a). Preference is given to polyglyceryl partial esters and sorbitan partial esters which contain, as hydrophobic moieties, fatty acid radicals having a chain length of from ≧10 to ≦18 carbon atoms. Very particular preference is given to a combination of polyglyceryl laurates and sorbitan laurates.

For the emulsifier component (b) containing secondary cationic groups, it is possible in principle to use all emulsifiers or surfactants containing cationic groups known to those skilled in the art. Preference is given to compounds selected from at least one of the groups of:
b1) quaternary ammonium compounds of the general formula I 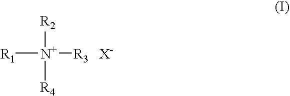
in which
R₁ is selected from the group of
branched or unbranced, cyclic or acyclic C₆₋₂₂-alkyl or -alkenyl radical,
R₅—COO—R₆— where R₅=branched or unbranched, cyclic or acyclic, optionally hydroxy-functional C₅₋₂₁-alkyl or -alkenyl radical and R₆=branched or unbranced C₂₋₅-alkylene radical,
R₅—CONH—R₆— where R₅ branched or unbranched, cyclic or acyclic, optionally hydroxy-functional C₅₋₂₁-alkyl or -alkenyl radical and R₆=branched or unbranched C₂₋₅-alkylene radical,
R₂, R₃, R₄ are each independently selected from the group of branched or unbranched, cyclic or acyclic C₆₋₂₂-allyl or -alkenyl radical,
R₅—COO—R₆— where R₅=branched or unbranched, cyclic or acyclic, optionally hydroxy-functional C₅₋₂-alkyl or -alkenyl radical and R₆=branched or unbranched C₂₋₅-alkylene radical,
R₅—CONH—R₆— where R₅=branched or unbranched, cyclic or acyclic, optionally hydroxy-functional C₅₋₂₁-alkyl or -alkenyl radical and R₆=branched or unbranched C₂₋₅-alkylene radical,
C₁₋₄-allyl radical,
C₂₋₄-alkylene radical,
H,
2-hydroxyethyl radical or 2-hydroxypropyl radical,
   X⁻ is an anion compatible with the quaternary ammonium compound, for example halide, methylsulfate, ethylsulfate, glycolate, lactate, acetate, sulfate, nitrate or phosphate,
   b2) pyridinium, oxazolinium or thiazolinium compounds containing one or more alkyl radicals having from ≧6 to ≦22 carbon atoms,
   b3) alkylguanidinium salts containing alkyl chains having from ≧6 to ≦22 carbon atoms,
   b4) benzylammonium compounds having one or more alkyl radicals having from ≧6 to ≦22 carbon atoms,
   b5) amine oxide salts containing one or more alkyl radicals having from ≧6 to ≦22 carbon atoms,
   b6) silicone-based polymers containing at least one quaternary ammonium group, a protonated amino group or an alkylguanidinium group,
   b7) compounds of the formula II 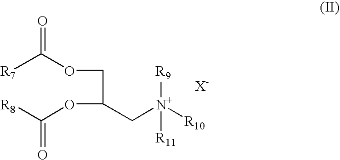
in which
R₇ and R₈ are each independently branched or unbranched, cyclic or acyclic C₆₋₂₂-alkyl or -alkenyl radicals,
A) R₉, R₁₀ and R₁₁ are each independently C₁₋₄-alkyl radicals,
   b8) compounds of the formula III 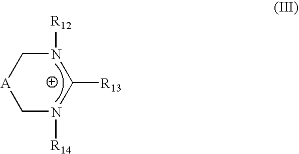
in which
A is a direct single or double bond,
R₁₂ is H or C₁₋₄-alkyl,
R₁₃ is H or C₁₋₂₂-alkyl,
R₁₄ is selected from the group of
H,
C₁₋₂₂-alkyl,
(CH₂)₂—NHCO—R₁₅ where R₁₅=branched or unbranched, cyclic or acyclic, optionally hydroxy-functional C₅₋₂₁-alkyl or -alkenyl radical,
COR₁₅ where R₁₅=branched or unbranched, cyclic or acyclic, optionally hydroxy-functional C₅₋₂₁-alkyl or -alkenyl radical,
   with the proviso that at least one of the R₁₂, R₁₃ and R₁₄ radicals contains at least 6 carbon atoms,
      b9) compounds of the formula IV 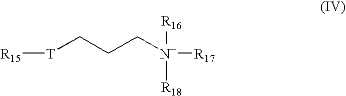
in which
R₁₅ is branched or unbranched C₆₋₂₂-alkyl or -alkenyl radical,
T is NH or O,
R₁₆, R₁₇, R₁₈ are each independently H, C₁₋₄-alkyl or 2-hydroxyalkyl,
   b10) salts of compounds of the formula R₉—O—(CH₂)₃—NH—(CH₂)₃—NH₂ where R₁₉=branched or unbranched C₆₋₂₂-alkyl or -alkenyl radical in which at least one of the two amine groups is present in protonated form,
   b11) compounds of the general formula V 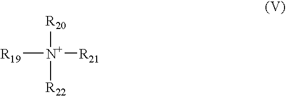
in which
R₁₉ is selected from
branched or unbranched C₆₋₂₂-alkyl or -alkenyl radical or
R₂₃—CONH—(CH₂)ₙ— where R₂₃=branched or unbranched C₅₋₂₁-alkyl or -alkenyl radical and n=2 to 4,
   R₂₀ and R₂₁ are each independently selected from H, C₁₋₄-alkyl radical,
   C₂₋₄-alkenyl radical or C₂₋₃-hydroxyalkyl radical and
   R₂₂ is selected from —CH₂COO⁻, —(CH₂)₂COO⁻, —(CH₂)₃SO₃⁻ and —CH₂CH₂OHCH₂ SO₃⁻.

Particularly preferred emulsifier components b) containing secondary cationic groups are cetrimonium chloride, behentrimonium chloride, behentrimonium methosulfate, dicetyldimonium chloride, distearyldimonium chloride, palmitamidopropyltrimonium chloride, quaternium-18, ricinolamidopropyltrimonium methosulfate, distearoylethyldimonium chloride, distearoylethylhydroxyethylmonium chloride or methosulfate, dioleoylethylhydroxyethylmonium chloride or methosulfate, dipalmitoylethylhydroxyethylmonium chloride or methosulfate, cocamido-propylbetaine, cocamphoacetate, and also cationic polymeric silicone compounds, for instance Quaternim-80 (e.g. ABIL® Quat 3272 (Degussa)) or mixtures of these compounds.

In the context of the present invention, cosurfactants are understood to mean those compounds which feature interface activity, which can be manifested in the lowering of interface tensions or the incorporation into interface films, but without these substances taken alone exhibiting the aggregation typical of surfactants to give micellar structures in water or the stabilization typical of emulsifiers for emulsion droplets.

In the context of the present invention, cosurfactants are additionally or alternatively understood to mean those compounds which feature an octanol-water partition coefficient log P or log K_{ow} which is between 0.8 and 2.2 and preferably between 1 and 2. The octanol-water partition coefficient is calculated from the decadic logarithm of the quotient of the amount of a substance dissolved in octanol and in water in equilibrium at room temperature (see: O. Fränzle, M. Straskraba in Ullman's Encyclopedia of Industrial Chemistry, 6^{th} edition, Wiley-VCH, Weinheim, 2000).

Advantageously, the inventive cosurfactants are nonionic organic compounds which have from 4 to 14 carbon atoms and one or more polar groups in the molecule.

Typical known nonaromatic cosurfactants are aliphatic alcohols such as butanol, pentanol, hexanol, heptanol, octanol, hexanediol or octanediol. In a preferred embodiment of the invention, the cosurfactants used are n-pentanol, n-hexanol, 1,2-hexanediol, 1,2-heptanediol or 1,2-octanediol.

In addition, the cosurfactants used are preferably also monoalkyl ethers or monoalkyl esters based on glycerol, ethylene glycol, propylene glycol or diethylene glycol with fatty acids or alcohols having from 6 to 10 carbon atoms.

In a preferred embodiment of the invention, the cosurfactants used are aromatic cosurfactants. In the context of the present invention, aromatic cosurfactants are understood to mean interface-active substances which contain one or more aryl groups and which, taken alone, do not form any micellar structures in water.

Advantageously, these aromatic cosurfactants additionally feature antimicrobial properties, i.e., they are aromatic cosurfactants with preservative properties. The use of such cosurfactants enables the preparation of inventive O/W emulsions which ideally need no further preservatives. In addition, it is of course possible to add further customary preservatives (as assistants and additives), as described, for instance, in DE 102005011785.6.

Aromatic cosurfactants which have preservative properties and are particularly preferred in accordance with the invention are phenoxyethanol, phenoxy-isopropanol and benzyl alcohol, taken alone or in combination with one or more alkylparaben esters, preferably methylparaben, ethylparaben, propylparaben, isopropylparaben, butylparaben. Particular preference is given to the use of mixtures of alkylparaben esters and phenoxyethanol, as are commercially available under the trade names Euxyl® K 300 (Schülke & Mayr) or Phenonip® (Clariant).

As mentioned, it is also possible to use mixtures of said preservation-active aromatic cosurfactants with other cosurfactants. For example, it is also possible to use a mixture of phenoxyethanol and ethylhexyl glycerol, as is commercially available under the name Euxyl® PE 9010 (Schülke & Mayr). Furthermore, preference may be given to using mixtures of phenoxyethanol and 1,2-octanediol (capryl glycol).

In the context of the present invention, oils are understood to mean compounds selected from the group of Guerbet alcohols based on fatty alcohols having from 6 to 20, preferably from 8 to 10 carbon atoms, esters of linear C₁-C₄₄-fatty acids with linear C₁-C₂₂-fatty alcohols, esters of branched C₁-C₄₄-carboxylic acids with linear C₁-C₂₂-fatty alcohols, esters of linear C₁-C₄₄-fatty acids with branched alcohols, esters of linear and/or branched fatty acids with polyhydric alcohols and/or Guerbet alcohols, triglycerides based on C₁-C₄₄-fatty acids, vegetable oils, branched primary alcohols, substituted cyclohexanes, Guerbet carbonates, dialk(en)yl ethers, dialk(en)yl carbonates and/or aliphatic or naphthenic hydrocarbons, silicone oils, dimethicones, cyclomethicones, ethoxylated and/or propoxylated organic alcohols, ethoxylated and/or propoxylated organic acids or mixtures thereof. Perfume oils known to those skilled in the art may also serve as the oil phases in the context of the invention.

In a preferred embodiment of the invention, the oils used are ester oils, ether-based oils, hydrocarbons, silicone oils, and mixtures of these compounds.

Useful ester oils include in particular mono- or diesters of linear and/or branched mono- and/or dicarboxylic acids having from ≧2 to ≦44 carbon atoms with linear and/or branched (especially 2-ethylhexanol), saturated or unsaturated alcohols having from ≧1 to ≦22 carbon atoms. Likewise suitable in the inventive context are the esterification products of aliphatic, difunctional or trifunctional alcohols (especially dimer diol and/or trimer diol) having from ≧2 to ≦36 carbon atoms with one or more monofunctional aliphatic carboxylic acids having from ≧1 to ≦22 carbon atoms. Also suitable in accordance with the invention are ester oils which contain aromatic groups.

The partial use of ester oils which have waxy character at room temperature, for instance myristyl myristate, can lead to a richer skin feel of the emulsions.

Useful ether oils are in particular dialkyl ethers having from ≧4 to ≦24 carbon atoms. Particularly suitable in accordance with the invention are saturated C₆-C₈-dialkyl ethers, for example di-n-octyl ether, di(2-ethylhexyl)ether, lauryl methyl ether or octyl butyl ether, and also didodecyl ether.

Particularly preferred oil components are the cosmetic ester oils ethylhexyl palmitate, ethylhexyl stearate, decyl cocoate, diethylhexyl carbonate, dioctyl carbonate, cetearyl ethylhexanoate, decyl oleate, isocetyl palmitate, cetearyl isononanoate, hexyl laurate, isopropyl isononanoate, isopropyl stearate, isopropyl palmitate, isopropyl myristate, isopropyl laurate and C₂₋₁₅ alkyl benzoate, and the cosmetic ether oil dicaprylyl ether and/or isohexadecane, paraffin oil, octyldodecanol and/or cyclopentasiloxane, and mixtures of the compounds mentioned.

In the context of the present invention, “polar solubilizers” are understood to mean polar compounds which are added in amounts of up to 10% by weight to the oil phases described below in order to obtain clear oil phases. The polar stabilizers are preferably water, glycols, polyalkylene glycols, glycerol polyglycerol or short-chain alcohols such as ethanol or isopropanol.

The assistants and additives used may be all assistants and additives known as prior art to a person skilled in the art, such as oils and waxes, commercial surfactants or emulsifiers, bodying agents, thickeners, for example based on polymer, inorganic and organic light protection filters, self-tanning agents, pigments, antioxidants, hydrotropes, active deodorant and antiperspirant ingredients, other active ingredients, dyes, additional preservatives and perfumes, as described, for instance, in DE 102005011785.6. The assistants and additives may be added either to the oil or to the water phase, or to the diluent water in the preparation process of the emulsion.

Preferred active ingredients are in particular tocopherol, tocopherol acetate, tocopherol palmitate, ascorbic acid, deoxyribonucleic acid, coenzyme Q10, retinol and retinyl derivatives, bisabolol, allantoin, phytantriol, panthenol, AHA acids, amino acids, hyaluronic acid, creatine (and creatine derivatives), creatinine, guanidine (and guanidine derivatives), ceramides, phytosphingosine (and phytosphingosine derivatives), sphingosine (and sphingosine derivatives), pseudoceramides, essential oils, peptides, protein hydrolyzates, salicylic acid, zinc ricinoleate, plant extracts and vitamin complexes.

The inventive O/W emulsions can in principle be prepared utilizing a simple stirrer apparatus. No additional homogenization step is required.

The preparation is preferably effected at room temperature by directly pouring a clear, monophasic oil phase comprising an emulsifier mixture, cosurfactants, oils and optionally customary assistants and additives into diluent water. In many cases, the preparation can also be effected in reverse, by adding the diluent water to the initially charged clear oil phase. If necessary, the oil phase may be converted to a clear phase by adding up to 10% by weight of a polar solubilizer. Such polar solubilizers may be water, glycols, polyalkylene glycols, glycerol, polyglycerols or short-chain alcohols such as ethanol or isopropanol. The polar solubilizer used is preferably water.

Homogeneous, clear oil phases are advantageous for the preparation of inventive fine O/W emulsions. The use of cloudy oil phases leads generally to relatively coarse emulsions whose long-term stability is often insufficient. The transition from clear to cloudy oil phases is fluid. The opacity at which emulsions with sufficient long-term stability can be prepared is dependent upon the type and amount of the components used and should be determined individually in these limiting cases.

Alternatively to the method mentioned, inventive fine oil-in-water emulsions can also be effected via the intermediate stage of a clear to transparent microemulsion-like concentrate. This concentrate consists generally of from >30 to ≦90% by weight of oil phase, preferably of from ≧40 to ≦80% by weight of oil phase, comprising an emulsifier mixture, cosurfactants, oils and optionally polar solubilizers and/or customary assistants and additives.

These clear to transparent microemulsion-like concentrates are preferably prepared at room temperature by stirring water into the oil phase. To prepare these concentrates, it is also possible to use cloudy oil phases. The optimal water content of the concentrates is formulation-dependent (for example on the oil used), but is generally from ≧10 to ≦70% by weight, preferably from ≧20 to ≦60% by weight.

These microemulsion-like concentrates may finally be diluted to give inventive oil-in-water emulsions. Both the preparation of the microemulsion-like concentrates and the final dilution step can be effected at room temperature using a simple stirrer apparatus.

The invention therefore further provides oil phases comprising:
A) an emulsifier mixture consisting of:
   a) at least one nonionic primary emulsifier, and
   b) at least one secondary emulsifier containing cationic groups;
B) one or more cosurfactants;
C) one or more oils;
D) from ≧0 to ≦10% by weight (based on the overall oil phase) of one or more polar solubilizers; and optionally
E) customary assistants and additives.

Preference is given in accordance with the invention to homogeneous and clear oil phases.

These oil phases can be prepared by the known prior art processes. For example, the oil phases, depending on the consistency and concentration of the components used, can be prepared by simply mixing the components at temperatures in the range of ≧20 to ≦75° C. These oil phases can be used at room temperature to prepare the inventive oil-in-water emulsions.

The invention further provides a process for preparing inventive oil-in-water emulsions, wherein these clear oil phases are preferably adjusted to a total water phase content of ≧70% by weight, preferably ≧80% by weight, at temperatures of ≦40° C., especially room temperature, with an appropriate water phase under conditions known per se.

The invention further provides clear to transparent microemulsion-like concentrates comprising:
A) an emulsifier mixture consisting of:
   a) at least one nonionic primary emulsifier, and
   b) at least one secondary emulsifier containing cationic groups;
B) one or more cosurfactants;
C) one or more oils; optionally
D) one or more polar solubilizers; and optionally
E) customary assistants and additives,
   with the proviso that the total water phase content of the microemulsion-like concentrates is from ≧10 to ≦70% by weight, based on the overall concentrate.

The invention further provides a process for preparing inventive oil-in-water emulsions, wherein these microemulsion-like concentrates are preferably adjusted to a total water phase content of ≧70% by weight, preferably ≧80% by weight, at temperatures of less than 40° C., especially room temperature, with an appropriate water phase under conditions known per se.

The invention further provides for the use of the inventive emulsions for producing cosmetic, dermatological or pharmaceutical formulations. In particular, the use as impregnating solutions for producing wet wipes, very particularly cosmetic wet wipes for the care and cleaning of the skin and skin appendages, are at the forefront.

The invention further provides for the use of the emulsions in cosmetic cleaning and care formulations for skin and skin appendages. In particular, the use in sprayable formulations is at the forefront, as used, for instance, for facecare and bodycare products, babycare, sun protection preparations, makeup removers and antiperspirants/deodorants, and for haircare.

The inventive oil-in-water emulsions are also outstandingly suitable both for the production of cleaning and care wet wipes and for direct use in the form of sprayable emulsion systems for the cleaning and care of surfaces in the household and industry, for example, textile care, leather care, the care and cleaning of metallic or nonmetallic surfaces, for example for the cleaning and care of automobiles or furniture.

The invention accordingly further provides for the use of the emulsions for producing cleaning compositions and care compositions for the household and industry, such as textiles, leather, plastics, metallic and nonmetallic surfaces. In particular, the use as impregnating solutions for producing wet wipes and the use in sprayable formulations are at the forefront.

The technical teaching described here enables, in a simple manner, the preparation at room temperature of PEG-free, low-viscosity, fine and prolonged-stability oil-in-water emulsions which already have sufficient preservation.

The secondary emulsifiers containing cationic groups used in this case can both cause a softening effect on the wet wipes and lead to better adhesion of the impregnating emulsion to skin and hair, and influence the sensory properties of the emulsions in a generally advantageous manner.

The examples which follow are intended to illustrate the subject matter of the invention in detail without restricting it to these examples. The concentrations in all examples are reported as % by weight.

The inventive oil phases, the inventive microemulsion-like concentrates and the inventive emulsions were prepared using a simple manual stirrer apparatus. After the corresponding oil phases had been prepared, both the conversion to inventive microemulsion-like concentrates and to inventive oil-in-water emulsions were effected at temperatures of <30° C.

Example Emulsifiers 1 to 9:

Description of the emulsifier systems used in the example formulations (the total percentages per emulsifier system in each case added up to 100):

| | |
|---|---|
| Emulsifier 1: | |
| Emulsifier component A: | 99.1% polyglyceryl-4 laurate¹⁾ |
| Emulsifier component B: | 0.9% cetyltrimonium chloride²⁾ |
| Emulsifier 2: | |
| Emulsifier component A: | 98.0% polyglyceryl-4 laurate¹⁾ |
| Emulsifier component B: | 2.0% quaternium-80³⁾ |
| Emulsifier 3: | |
| Emulsifier component A: | 79.4% polyglyceryl-4 laurate¹⁾ |
| | 19.8% sorbitan laurate⁴⁾ |
| Emulsifier component B: | 0.8% cetyltrimonium chloride²⁾ |
| Emulsifier 4: | |
| Emulsifier component A: | 79.2% polyglyceryl-4 laurate¹⁾ |
| | 19.8% sorbitan laurate⁴⁾ |
| Emulsifier component B: | 1.0% cetyltrimonium chloride²⁾ |
| Emulsifier 5: | |
| Emulsifier component A: | 99.4% polyglyceryl-4 laurate¹⁾ |
| Emulsifier component B: | 0.6% cetyltrimonium chloride²⁾ |
| Emulsifier 6: | |
| Emulsifier component A: | 96.6% polyglyceryl-4 laurate¹⁾ |
| Emulsifier component B: | 3.4% quaternium-80³⁾ |
| Emulsifier 7: | |
| Emulsifier component A: | 98.7% polyglyceryl-4 laurate¹⁾ |
| Emulsifier component B: | 1.3% palmitamidopropyltrimonium chloride⁵⁾ |
| Emulsifier 8: | |
| Emulsifier component A: | 68.9% polyglyceryl-4 laurate¹⁾ |
| | 29.5% sorbitan laurate⁴⁾ |
| Emulsifier component B: | 1.6% cetyltrimonium chloride²⁾ |
| Emulsifier 9: | |
| Emulsifier component A: | 99.3% polyglyceryl-4 laurate¹⁾ |
| Emulsifier component B: | 0.7% dicetyldimonium chloride⁶⁾ |

TEGO® Care PL 4 (Goldschmidt GmbH)
VARISOFT® 300 (Degussa)
ABIL® Quat 3272 (Degussa)
TEG® SML (Degussa)
VARISOFT® PATC (Degussa)
VARISOFT® 432 PPG (Degussa)

Examples of Clear Oil Phases 1 to 5:

These examples show in particular the composition of inventive clear oil phases which can be processed in a further step at room temperature to give inventive, fine oil-in-water emulsions (see example emulsions 1 to 5).

| | Clear oil phases | | | | |
|---|---|---|---|---|---|
| | 1 | 2 | 3 | 4 | 5 |
| | in % | in % | in % | in % | in % |
| Emulsifier 1 | 21.7 | | | | |
| Emulsifier 2 | | 21.4 | | | |
| Emulsifier 3 | | | 21.7 | | |
| Emulsifier 4 | | | | 21.7 | |
| Emulsifier 5 | | | | | 21.8 |
| isopropyl palmitate | | | | | 64.9 |
| ethylhexyl palmitate | | | | 64.4 | |
| diethylhexyl carbonate | 64.6 | 63.0 | | | |
| decyl cocoate | | | 64.7 | | |
| phenoxyethanol | 12.0 | 11.8 | 12.1 | 12.0 | 12.1 |
| water | 1.7 | 3.8 | 1.5 | 1.9 | 1.2 |

Examples of Clear to Transparent Microemulsion-Like Concentrates 1 to 5:

These examples show in particular the composition of inventive clear to transparent microemulsion-like concentrates which can be processed in a further step at room temperature to give inventive, fine oil-in-water emulsions (see example emulsions 6 to 10).

| | Microemulsion-like | | | | |
|---|---|---|---|---|---|
| | concentrates | | | | |
| | 1 | 2 | 3 | 4 | 5 |
| | in % | in % | in % | in % | in % |
| Emulsifier 1 | 11.0 | | | | |
| Emulsifier 6 | | 10.9 | | | |
| Emulsifier 7 | | | 11.0 | | |
| Emulsifier 8 | | | | 10.9 | |
| Emulsifier 9 | | | | | 12.0 |
| cetearyl isononanoate | | | | 32.3 | |
| isopropyl palmitate | | | | | 17.9 |
| paraffin oil | | | | | 17.9 |
| (25 mPas at 30° C.) | | | | | |
| diethylhexyl carbonate | 32.6 | 31.7 | 32.4 | | |
| phenoxyethanol | 6.1 | 5.9 | 6.1 | 6.0 | 1.9 |
| capryl glycol⁷⁾ | | | | | 1.9 |
| water | 50.3 | 51.5 | 50.5 | 50.8 | 48.4 |

⁷⁾Dermosoft ® Octiol (Dr. Straetmans) (1,2-octanediol)

Example Emulsions 1 to 10:

Emulsions 1 to 10 illustrate the structure of inventive emulsions by way of example.

Emulsions 1 to 5 were prepared by pouring the clear oil phases (examples 1 to 5 (see above)) into water at room temperature using a simple manual stirrer apparatus.

Emulsions 6 to 10 were likewise prepared at room temperature by diluting the clear to transparent microemulsion-like concentrates (corresponding examples 1 to 5 (see above)) using a simple manual stirrer apparatus.

Example emulsions 1 and 6 are identical in this composition. Only the preparation of the emulsions was effected in different ways (1: directly from dilution of the oil phase; 6: via the microemulsion-like concentrate). Viscosity, blue mold, and stability of the two emulsion examples were absolutely comparable.

All example emulsions were low in viscosity, had fine distribution and had long-term stability.

| | Example emulsions | | | | |
|---|---|---|---|---|---|
| | 1 | 2 | 3 | 4 | 5 |
| | in % | in % | in % | in % | in % |
| Emulsifier 1 | 1.3 | | | | |
| Emulsifier 2 | | 1.3 | | | |
| Emulsifier 3 | | | 1.3 | | |
| Emulsifier 4 | | | | 1.3 | |
| Emulsifier 5 | | | | | 1.3 |
| isopropyl palmitate | | | | | 3.7 |
| ethylhexyl palmitate | | | | 3.7 | |
| diethylhexyl carbonate | 3.7 | 3.7 | | | |
| decyl cocoate | | | 3.7 | | |
| phenoxyethanol | 0.7 | 0.7 | 0.7 | 0.7 | 0.7 |
| water | 94.3 | 94.3 | 94.3 | 94.3 | 94.3 |

| | Example emulsions | | | | |
|---|---|---|---|---|---|
| | 6 | 7 | 8 | 9 | 10 |
| | in % | in % | in % | in % | in % |
| Emulsifier 1 | 1.3 | | | | |
| Emulsifier 6 | | 1.3 | | | |
| Emulsifier 7 | | | 1.3 | | |
| Emulsifier 8 | | | | 1.3 | |
| Emulsifier 9 | | | | | 5.0 |
| cetearyl isononanoate | | | | 3.7 | |
| isopropyl palmitate | | | | | 7.5 |
| paraffin oil | | | | | 7.5 |
| (25 mPas at 30° C.) | | | | | |
| diethylhexyl carbonate | 3.7 | 3.7 | 3.7 | | |
| phenoxyethanol | 0.7 | 0.7 | 0.7 | 0.7 | 0.8 |
| capryl glycol⁷⁾ | | | | | 0.8 |
| water | 94.3 | 94.3 | 94.3 | 94.3 | 78.4 |

Particle Size Determination by Dynamic Light Scattering:

By way of example for the extremely fine degree of dispersion of the inventive emulsions, the particle size of individual example emulsions was characterized with the aid of dynamic light scattering.

Dynamic light scattering is based on the analysis of the variations of the scattered light intensity of diffusing particles in solution. For dilute solutions, it is possible thereby to determine the coefficient of diffusion of the particles in solution, which can be converted via the Stokes-Einstein equation to a mean hydrodynamic radius <rₕ> of the particles (here: emulsion droplets).

In the present case, the example emulsions mentioned were diluted with demineralized water to an oil phase content of, in each case, 0.5% and characterized with the aid of a dynamic light scattering instrument from Malvern Instruments (HPPS 3.1) at 25° C. In each case, the intensity-weighted mean values of three measurements were reported with 100 seconds of measurement time.

The complete intensity-weighted radius distributions of the selected example emulsions are shown in the sole drawing FIGURE that accompanys the present application. The following table provides a tabulated form of the information provided by the sole drawing FIGURE of the present application.

| Example | |
|---|---|
| emulsion | <rₕ> in nm |
| 2 | 50 |
| 3 | 90 |
| 4 | 60 |
| 5 | 50 |
| 9 | 55 |

While the invention has been described herein with reference to specific embodiments, features and aspects, it will be recognized that the invention is not thus limited, but rather extends in utility to other modifications, variations, applications, and embodiments, and accordingly all such other modifications, variations, applications, and embodiments are to be regarded as being within the spirit and scope of the invention.

What is claimed is:

1. A prolonged-stability, low-viscosity, fine oil-in-water emulsion comprising:
A) an emulsifier mixture consisting of
a) at least one nonionic primary emulsifier, and
b) at least one secondary emulsifier containing cationic groups;
B) one or more cosurfactants; and
C) one or more oils, with the proviso that the emulsion has a water phase content that is ≧70% by weight based on the overall emulsion.

2. The oil-in-water emulsion as claimed in claim 1, which has a water phase content of from ≧80 to ≦99% by weight.

3. The oil-in-water emulsion as claimed in claim 1, wherein emulsifier mixture (A), cosurfactants (B) and oils (C) are present in proportions by weight of from ≧10 to ≦30 (A), from ≧1 to ≦20 (B) and from ≧50 to ≦89 (C) based on the sum of these components.

4. The oil-in-water emulsion as claimed in claim 1, wherein the emulsifier mixture (A) is composed of from ≧75 to ≦99.9% by weight of nonionic primary emulsifier component (a) and from ≧0.1 to ≦25% by weight of secondary emulsifier component (b) containing cationic groups, based on the sum of these components.

5. The oil-in-water emulsion as claimed in claim 1, wherein the primary nonionic emulsifier component (a) in the emulsifier mixture (A) comprises one or more emulsifiers selected from the following groups
a1) glyceryl and polyglyceryl partial esters,
a2) sorbitan or sorbitol partial esters,
a3) carbohydrate esters,
a4) (alkylpoly)glycosides, and
mixtures of a1)-a4).

6. The oil-in-water emulsion as claimed in claim 1, wherein the primary nonionic emulsifier component (a) consists of a mixture of from ≧25 to ≦100% by weight of polyglyceryl partial esters and from ≧0 to ≦75% by weight of sorbitan esters which contain, as hydrophobic moieties, aliphatic, linear or branched, optionally unsaturated and/or hydroxy-functionalized carboxylic acids having a chain length of from ≧8 to ≦18 carbon atoms.

7. The oil-in-water emulsion as claimed in claim 1, wherein the primary nonionic emulsifier component (a) consists of polyglyceryl laurates or mixtures of polyglyceryl laurates with sorbitan laurates.

8. The oil-in-water emulsion as claimed in claim 1, wherein the secondary emulsifier component (b) containing cationic groups comprises one or more emulsifiers which are selected from at least one of the groups of
b1) quaternary ammonium compounds of the general formula I 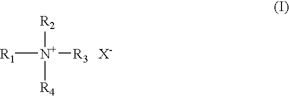
in which
R₁ is selected from the group of
branched or unbranced, cyclic or acyclic C₆₋₂₂-alkyl or -alkenyl radical,
R₅—COO—R₆— where R₅ branched or unbranched, cyclic or acyclic, optionally hydroxy-functional C₅₋₂₁-alkyl or -alkenyl radical and R₆=branched or unbranced C₂₋₅-alkylene radical,
R₅—CONH—R₆— where R₅ branched or unbranched, cyclic or acyclic, optionally hydroxy-functional C₅₋₂₁ alkyl or -alkenyl radical and R=branched or unbranched C₂₋₅-alkylene radical,
R₂, R₃, R₄ are each independently selected from the group of
branched or unbranched, cyclic or acyclic C₆₋₂₂-alkyl or -alkenyl radical,
R₅—COO—R₆— where R₅=branched or unbranched, cyclic or acyclic, optionally hydroxy-functional C₅₋₂₁-alkyl or -alkenyl radical and R₆=branched or unbranched C₂₋₅-alkylene radical,
R₅—CONH—R₆— where R₅=branched or unbranched, cyclic or acyclic, optionally hydroxy-functional C₅₋₂₁-alkyl or -alkenyl radical and R₆=branched or unbranched C₂₋₅-alkylene radical,
C₁₋₄-alkyl radical,
C₂₋₄-alkylene radical,
H,
2-hydroxyethyl radical or 2-hydroxy-propyl radical,
X⁻ is an anion compatible with the quaternary ammonium compound,
b2) pyridinium, oxazolinium or thiazolinium compounds containing one or more alkyl radicals having from ≧6 to ≦22 carbon atoms,
b3) alkylguanidinium salts containing alkyl chains having from ≧6 to ≦22 carbon atoms,
b4) benzylammonium compounds having one or more alkyl radicals having from ≧6 to ≦22 carbon atoms,
b5) amine oxide salts containing one or more alkyl radicals having from ≧6 to ≦22 carbon atoms,
b6) silicone-based polymers containing at least one quaternary ammonium group, a protonated amino group or an alkylguanidinium group,
b7) compounds of the formula II 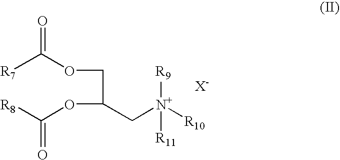
in which
R₇ and R₈ are each independently branched or unbranched, cyclic or acyclic C₆₋₂₂-alkyl or -alkenyl radicals,
R₉, R₁₀ and R₁₁ are each independently C₁₋₄-alkyl radicals,
b8) compounds of the formula III 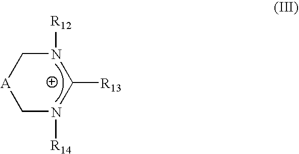
in which
A is a direct single or double bond,
R₁₂ is H or C₁₋₄-alkyl,
R₁₃ is H or C₁₋₂₂-alkyl,
R₁₄ is selected from the group of
H,
C₁₋₂₂-alkyl,
(CH₂)₂—NHCO—R₁₅ where R₁₅=branched or unbranched, cyclic or acyclic, optionally hydroxy-functional C₅₋₂₁-alkyl or -alkenyl radical,
COR₁₅ where R₁₅ branched or unbranched, cyclic or acyclic, optionally hydroxy-functional C₅₋₂₁-alkyl or -alkenyl radical,
with the proviso that at least one of the R₁₂, R₁₃ and R₁₄ radicals contains at least 6 carbon atoms,
b9) compounds of the formula IV 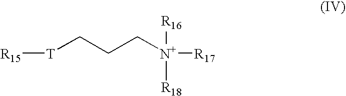
in which
R₁₅ is branched or unbranched C₆₋₂₂-alkyl or -alkenyl radical,
T is NH or O,
R₁₆, R₁₇, R₁₈ are each independently C₁₋₄-alkyl or 2-hydroxyalkyl,
b10) salts of compounds of the formula R₁₉—O—(CH₂)₃—NH—(CH₂)₃—NH₂ where R₁₉=branched or unbranched C₆₋₂₂-alkyl or -alkenyl radical in which at least one of the two amine groups is present in protonated form,
b11) compounds of the general formula V 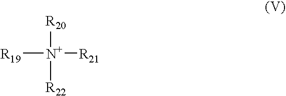
in which
R₁₉ is selected from
branched or unbranched C₆₋₂₂-alkyl or -alkenyl radical or
R₂₃—CONH—(CH₂)ₙ— where R₂₃ branched or unbranched C₅₋₂₁-alkyl or -alkenyl radical and n=2 to 4,
R₂₀ and R₂₁ are each independently selected from H, C₁₋₄-alkyl radical, C₂₋₄-alkenyl radical or C₂₋₃-hydroxyalkyl radical and
R₂₂ is selected from —CH₂COO⁻, —(CH₂)₂COO⁻, —(CH₂)₃SO₃⁻ and —CH₂CH₂OHCH₂SO₃⁻.

9. The oil-in-water emulsion as claimed in claim 1, wherein said one or more cosurfactants is at least one aliphatic cosurfactant selected from n-pentanol, n-hexanol, 1,2-hexanediol, 1,2-heptanediol and 1,2-octanediol.

10. The oil-in-water emulsion as claimed in claim 1, wherein said at least one cosurfactant is an aromatic cosurfactant.

11. The oil-in-water emulsion as claimed in claim 10, wherein the aromatic cosurfactant is one of phenoxyethanol, phenoxyisopropanol, benzyl alcohol, and alkylparaben esters alone or in mixtures with one another or in mixtures with aliphatic cosurfactants.

12. The oil-in-water emulsion as claimed in claim 1, wherein the one or more oils include cosmetic ester oils, ether oils or mineral oils.

13. The oil-in-water emulsion as claimed in claim 1, wherein the one or more oils is at least one compound selected from the group of ethylhexyl palmitate, ethylhexyl stearate, decyl cocoate, diethylhexyl carbonate, dioctyl carbonate, cetearyl ethylhexanoate, decyl oleate, isocetyl palmitate, cetearyl isononanoate, hexyl laurate, isopropyl isononanoate, isopropyl stearate, isopropyl palmitate, isopropyl myristate, isopropyl laurate, C₁₂₋₁₅ alkyl benzoate, dicaprylyl ether, mineral oil, isohexadecane, cyclopentasiloxane, octyldodecanol and mixtures of these compounds.

14. The oil-in-water emulsion as claimed in claim 1, further comprising one of one or more polar solubilizers and one or more customary assistants or additives.

15. An oil phase comprising:
A) an emulsifier mixture consisting of
a) at least one nonionic primary emulsifier, and
b) at least one secondary emulsifier containing cationic groups;
B) one or more cosurfactants;
C) one or more oils; and
D) from ≧0 to ≦10% by weight, based on the overall oil phase, of one or more polar solubilizers.

16. The oil phase as claimed in claim 15 further comprising one or more customary assistants or additives.

17. The oil phase as claimed in claim 15, which is homogeneous and clear.

18. A process for preparing oil-in-water emulsions, which comprises adjusting an oil phase as claimed in claim 15 to a total water phase content of ≧70% by weight with an appropriate water phase.

19. A clear to transparent microemulsion-like concentrate comprising:
A) an emulsifier mixture consisting of
a) at least one nonionic primary emulsifier, and
b) at least one secondary emulsifier containing cationic groups;
B) one or more cosurfactants; and
C) one or more oils,
with the proviso that the microemulsion-like concentrate has a total water phase from ≧10 to ≦70% by weight, based on the overall concentrate.

20. A process for preparing oil-in-water emulsions, which comprises adjusting clear to transparent microemulsion-like concentrates as claimed in claim 19 to a total water phase content of ≧70% by weight with an appropriate water phase.
